(19) United States
(12) Patent Application Publication
(10) Pub. No.: US 2025/0387256 A1
(43) Pub. Date: Dec. 25, 2025

(54) HOT STONE MASSAGE SYSTEM

(71) Applicant: Jamazing Products LLC, St. Augustine, FL (US)

(72) Inventor: Jason Baggott, St. Augustine, FL (US)

(21) Appl. No.: 18/747,664

(22) Filed: Jun. 19, 2024

(51) Int. Cl. A61F 7/00

(52) U.S. Cl. CPC A61F 7/007; A61F 2007/0078; A61F 2007/0095

(57) ABSTRACT

A massage system includes a case defining at least one cavity and at least one stone removably arranged inside the at least one cavity. The at least one stone includes a housing and at least one heater arranged inside the housing and adapted to heat at least a portion of the housing. The at least one also includes at least one battery disposed inside the housing and electrically coupled to the at least one heater to power the at least one heater. The massage system further includes a controller mounted to the case and adapted to control the at least one heater to achieve a desired heating of the at least one stone.

### TECHNICAL FIELD

The present disclosure relates, generally, to a massage system, and more particularly relates to a massage system having a stone for massaging a body of a person and a battery to power a heater of the stone.

### BACKGROUND

Generally, in a hot stone massage therapy, stones are heated, and then appropriate stone pressure is applied to the body by massage. Hot stone massage can be effective to improve body circulation and promote relaxation. However, traditional methods for heating the stone are not effective, and the stones will quickly lose their heat limiting the desired duration of the massage therapy. To overcome this challenge, the therapists, generally, uses a large number of massage stones to ensure successful use of the massage stones. This adds to overall cost and complexity to the stone massage therapy.

### SUMMARY

In one aspect of the disclosure, a massage system is disclosed. The massage system includes a case defining at least one cavity and at least one stone removably arranged inside the at least one cavity. The at least one stone includes a housing and at least one heater arranged inside the housing and adapted to heat at least a portion of the housing. The at least one also includes at least one battery disposed inside the housing and electrically coupled to the at least one heater to power the at least one heater. The massage system further includes a controller mounted to the case and adapted to control the at least one heater to achieve a desired heating of the at least one stone.

In some additional, alternative, or selectively cumulative embodiments, the at least one heater includes a first heater arranged inside the housing to heat a flat first surface of the housing.

In some additional, alternative, or selectively cumulative embodiments, the first heater is a ceramic heater including a ceramic plate.

In some additional, alternative, or selectively cumulative embodiments, the at least one heater includes a second heater arranged inside the housing to heat a central circumferential surface of the housing.

In some additional, alternative, or selectively cumulative embodiments, the second heater is a coil heater including a coil heating element.

In some embodiments, the second heater is a polyimide heater, a ceramic heater, or any other type of suitable heater known in the art.

In some additional, alternative, or selectively cumulative embodiments, the massage system further includes at least one display mounted to the case and communicatively coupled to the controller and configured to display a temperature of the at least one stone.

In some additional, alternative, or selectively cumulative embodiments, the massage system further includes at least one selector associated with the at least one stone and configured to enable a user to select a desired temperature of the at least one stone. The controller controls the at least one heater based on an input received from the at least one selector.

In some additional, alternative, or selectively cumulative embodiments, the massage system further includes a first communication device mounted to the case and a second communication device arranged inside the housing of the at least one stone to enable a data transfer between the controller and the at least one stone to enable the controller to control of the at least one stone.

In some additional, alternative, or selectively cumulative embodiments, the at least one stone includes a plurality of indicator lights mounted to the housing and configured to be actuated, by the controller, based one or more operating conditions of the at least one stone.

In some additional, alternative, or selectively cumulative embodiments, the controller actuates and control the indicator lights to emit one or more colors based on the one or more operating conditions of the at least one stone.

In some additional, alternative, or selectively cumulative embodiments, the housing includes a frame supporting the at least one battery and the at least one heater, and a pair of covers coupled to the frame.

In some additional, alternative, or selectively cumulative embodiments, each of the pair of covers is removably engaged to the frame.

In some additional, alternative, or selectively cumulative embodiment, the frame includes a base, a first wall extending in a first direction from the base and defining a receptacle to receive the at least one battery, and a second wall extending in a second direction, opposite to the first direction, from the base and defining a compartment to receive and support the at least one heater.

In some additional, alternative, or selectively cumulative embodiment, a first cover of the pair of covers is arranged covering the receptacle, and a second cover of the pair of covers is arranged covering the compartment.

In some additional, alternative, or selectively cumulative embodiment, a first heater of the at least one heater is arranged partially inside the compartment and extends outwardly of the compartment to heat a flat first surface of the second cover. Also, a second heater of the at least one heater is wrapped circumferentially around an outer surface of the second wall to heat a central circumferential surface of the housing.

### BRIEF DESCRIPTION OF THE DRAWINGS

FIG. 1 shows a top perspective view of a massage system having a case and two massage stones arranged inside the case, in accordance with an embodiment of the present disclosure;

FIG. 2 shows a top perspective view of the massage system of FIG. 1 with a first stone arranged outside the case, in accordance with an embodiment of the present disclosure;

FIG. 3 shows a top perspective view of the first stone, in accordance with an embodiment of the present disclosure;

FIG. 4 shows an exploded view of the first stone of FIG. 3, in accordance with an embodiment of the present disclosure; and

FIG. 5 shows a partial exploded view of the first stone of FIG. 3 with a lower cover of the first stone removed from a frame of the first stone and depicting two heaters of the first stone, in accordance with an embodiment of the present disclosure.

### DETAILED DESCRIPTION

The present disclosure is directed to massage system having heated stones that are used for massaging a body of a person. The stones of the massage system include batteries to suitably heat the stones to a desired temperature range to provide effective massage. Moreover, each stone includes a heated central circumferential i.e., cylindrical surface that can be rolled on a body part of the person. As the surface area that is in contact with the body of the person is smaller, the massaging of the body via the circumferential surface, the stone applies relatively larger pressure on the area, providing a deep massage therapy.

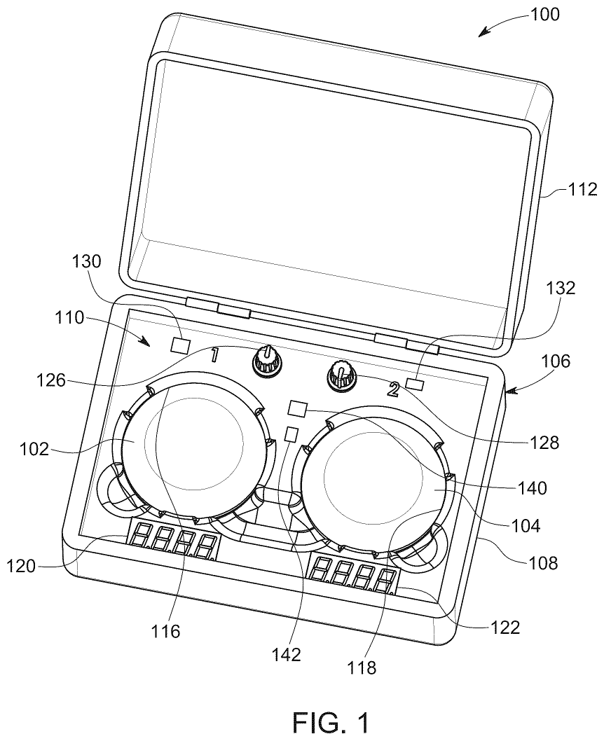

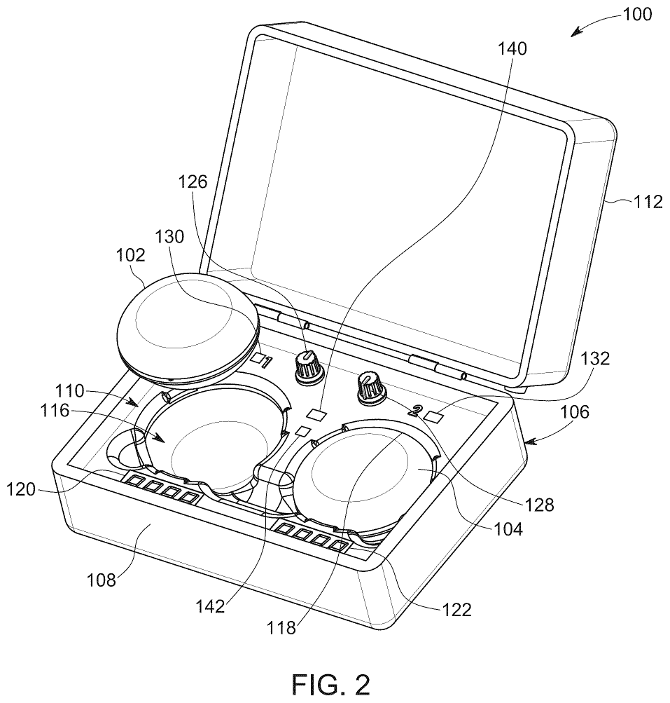

Referring to FIGS. 1 and 2, a massage system 100 for massaging a body of a person is shown. The massage system 100 includes at least one stone, for example, a first stone 102 and a second stone 104, for performing the massage, and a case 106 to store the at least one stone 102, 104. As shown, the case 106 includes a casing 108 defining a chamber 110 to receive and store the stones 102, 104 and various other component of the system 100, and a lid 112 pivotally connected to the casing and configured to pivot between a first position i.e., open position and a second position i.e., closed position. In the first position, the lid 112 is arranged relatively away from chamber 110 and facilitates an access of the inside of the chamber 110, while in the second position, the lid 112 is disposed covering the chamber 110.

As shown, the chamber 110 includes two cavities 116, 118 separated from each other by a partition wall to receive and hold the two stones 102, 104. In the illustrated embodiment, the cavities 116, 118 include substantially hemispherical shape. However, the cavities 116, 118 may include a shape and structure complimentary to that of the stones 102, 104 to enable a proper insertion and retention of the stones 102, 104 inside the cavities 116, 118. Moreover, the system 100 includes at least one display, for example, a first display 120 and a second display 122, arranged inside the chamber 110 and mounted to the casing 108. The first display 120 and the second display 122 are arranged in communication with the first stone 102 and the second stone 104 respectively, and are configured to display a temperature of the first stone 102 and the second stone 104, respectively. It may be appreciated that the displays 120, 122 may be configured to display the temperatures of the respective stones 102, 104 only when the stones 102, 104 are either arranged inside the respective cavities 116, 118 and/or are disposed/present within a predefined distance from the case 106.

Moreover, the system 100 includes at least one selector, for example, a first selector 126 and a second selector 128, disposed inside the chamber 110 and mounted to the casing 108 and configured to enable a user to select a desired temperature of the at least one stone, for example, the first stone 102 and the second stone 104. In the illustrated embodiment, the first selector 126 enables a user to select a desired temperature of the first stone 102, while a second selector 128 facilitates the user to select a desired temperature of the second stone 104.

In some embodiments, the first stone 102 and the second stone 104 are controlled to achieve the selected temperature only when the stones 102, 104 are arranged inside the respective cavities 116, 118. To enable such configuration and control, the system 100 includes at least one sensor, for example, a first sensor 130 and a second sensor 132, to respectively determine a presence or an absence of the first stone 102 inside a first cavity 116 and second stone 104 inside a second cavity 118. Based on the input from the sensors 130, 132, a controller 140 of the massage system 100 determines the presence of the stones 102, 104 inside the respective cavities 116, 118, and controls the temperatures of the stones 102, 104 to achieve the selected temperature.

In some embodiments, the controller 140 is configured to interact with the stones 102, 104 when the stones are arranged outside the cavities 116, 118 but within a predefined distance from the case 106. In such a case, the controller 140 is adapted to the control the temperature of the stones 102, 104 based on the input from the selectors 126, 128 to achieve the desired temperature even when the stones 102, 104 are arranged outside the cavities 116, 118. In the illustrated embodiment, the controller 140 is arranged inside the chamber 110 and is mounted to the casing 108. The controller 140 is communicatively coupled to the sensors 130, 132, the selectors 126, 128, the stones 102, 104, and other electrical and electronic components of the system 100. Further, the controller 140 is configured to assign/pair the first stone 102 with the first cavity 116, and the second stone 104 with the second cavity 118, accordingly assigns the first sensor 130, the first display 120 and the first selector 126 to the control and functioning of the first stone 102 and assigns the second sensor 132, the second display 122, and the second selector 128 to the control and functioning of the second stone 104.

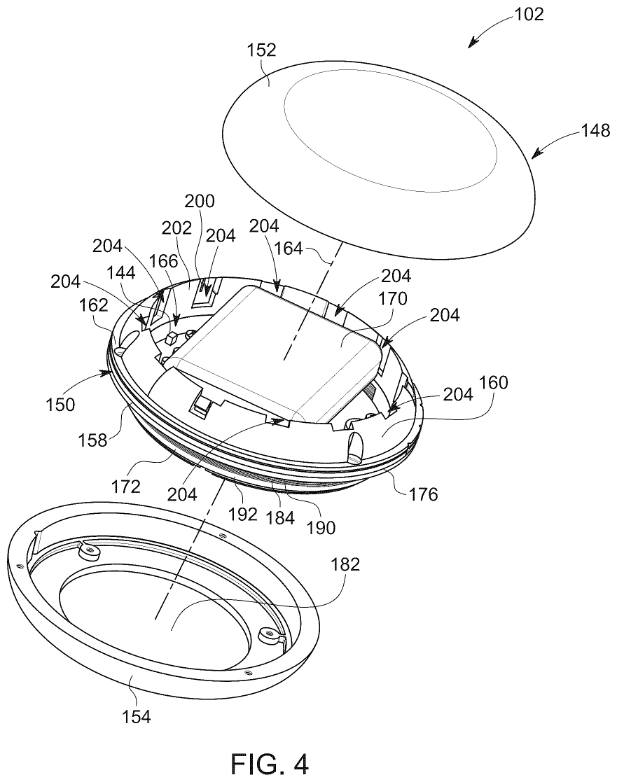

To facilitate the pairing/assigning of the first stone 102 with the first cavity 116 and the second stone 104 with the second cavity 118, the system 100 includes a first communication device 142 arranged inside the casing 108, while each of the stones 102, 104 includes a second communication device 144 (shown in FIG. 4) arranged to communicate with the first communication device 142 and facilitates a data exchange between the controller 140 and the stones 102, 104. In an embodiment, each of the stones 102 includes an identification code and the controller 140 receives the identification codes via the first communication device 142 and the second communication device 144, and identifies the stones 102, 104 as the first stone 102 and the second stone 104 based on the identification codes. It may be appreciated that the controller 140 based on the pairing, the controller 140 enables the various respective components of the system 100, for example, the displays, the sensors, the selectors, etc., with the functions and controls of the first stone 102 and the second stone 104. It may be appreciated that first and second communication devices 142, 144, each may include short range communication device, such as, but not limited to, Bluetooth™ devices, near field communication devices, radio frequency communication devices, or any other suitable communication devices, known in the art, adapted to enable data exchange between the controller 140 and each of the stones 102, 104. It may be appreciated both the first stone 102 and the second stone 104 are similar in structure, construction, assembly, and functionality, and therefore, for the sake of clarity and brevity, the structure, the construction, the assembly, and the functionality of the stones 102, 104 are described with reference to a single stone, for example, the first stone 102.

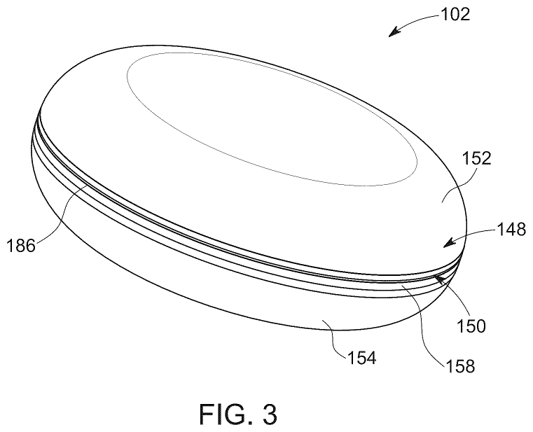

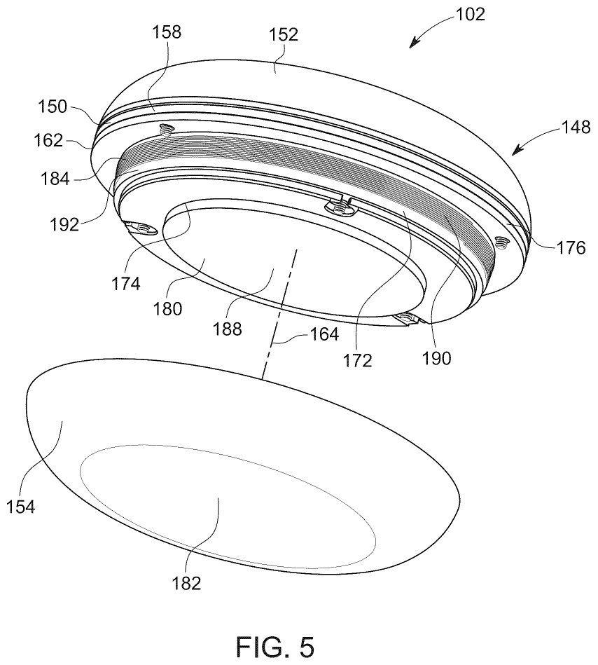

As shown in FIGS. 3 to 5, the first stone 102 includes a housing 148 having a frame 150 to support various components of the first stone 102, a first cover 152 hereinafter referred to as an upper cover 152 having covering a top half of the frame 150, and a second cover 154, hereinafter referred to as a lower cover 154, to cover a lower half of the frame 150. As shown in FIG. 4, each of the upper and lower covers 152, 154 includes a bowl shape having a flat bottom. Further, referring to FIGS. 4 and 5, the frame 150 includes a base 158 having a substantially circular disc shaped structure, and a wall 160, i.e., first wall 160, best shown in FIG. 4, extending vertically from a location proximate to an outer edge 162 of the base 158 and arranged circularly around a central axis 164 of the base 158. The first wall 160 defines a receptacle 166 to store and hold at least one battery 170 (shown in FIG. 4) of the first stone 102 to power various electrical and/or electronic component of the first stone 102. In some embodiments, the at least one battery 170 is a rechargeable battery. Alternatively, the at least one battery 170 may be a non-rechargeable battery.

Also, as shown in FIG. 5, the frame includes a second wall 172 extending vertically from the base 158 in a direction opposite to a direction of extension of the first wall 160 from the base 158. As with the first wall 160, the second wall 172 is arranged circularly around the central axis 164 of the base 158 and defining a compartment 174. Accordingly, the first wall 160 is arranged on a first side of the base 158, while the second wall 172 is disposed on a second side of the base 158. As shown in FIG. 5, the second wall 172 is arranged at a radial offset from a circumferential surface 176 of the base 158, and is arranged radially inwardly from the circumferential surface 176. An opening of the compartment 174 is covered by the flat bottom of the lower cover 154, while the receptacle 166 is covered by the upper cover 152 in the assembly of the covers 152, 154 with the frame 150. It may be appreciated that the covers 152, 154 may be removably engaged with the frame 150 to allow am access of the compartment 174 and the receptacle 166 arranged inside the compartment 174 and the receptacle 166 and/or any other components of the first stone 102 mounted to the frame 150.

Further, the first stone 102 includes at least one heater to heat at least a portion of the housing 148 of the first stone 102. In the illustrated embodiment, the at least one heater includes a first heater 180, best shown in FIG. 5, arranged to heat a flat bottom/first surface 182 of the lower cover 154, and a second heater 184 arranged to heat a central circumferential/cylindrical surface/portion 186, best shown in FIG. 3, of the housing 148 of the first stone 102. The central circumferential surface/portion 186 includes a substantial cylindrical surface defined at an interface of the lower cover 154, the upper cover 152 and the base 158. In the embodiment, the first heater 180 is a ceramic heater having a ceramic plate 188, shown in FIG. 5, arranged, at least partially inside the compartment 174 such that a portion of the ceramic plate 188 extends outwardly of the compartment 174 in the axial direction. The ceramic plate 188 is arranged to the contact an inner surface of the flat bottom surface of the lower cover 154 when the lower cover 154 is arranged assembled with the frame 150, covering the ceramic plate 188 and the second wall 172 and attached to the base 158 of the frame 150. In an embodiment, the lower cover 154 and the upper cover 152 are in threaded engagement with frame 150. However, the covers 152, 154 may be press fitted or the adhesively engaged with the frame 150. Additionally, the stone 102 includes suitable seals or gaskets that is arranged at the interface of frame and the covers to prevent an ingress of sweat, water, and oils commonly encountered during massage sessions.

Moreover, the second heater 184 is a coil heater, best shown in FIG. 5, having a coil type heating element 190 arranged/wrapped circumferentially around a portion of an outer surface 192 of the second wall 172. Although the second heater 184 is shown and contemplated as the coil heater, it may be appreciated that the second heater 184 may be a polyimide heater, a ceramic heater, or any other suitable heater known in the art. The second heater 184 facilitates a heating of circumferential central portion 186 of the first stone 102 to enable the massage a body part of person by rolling the first stone 102 on the body part. Accordingly, the second heater 184 facilitates in providing effective massage to difficult to reach areas, for example, a side portion of the neck of the person and/or providing a concentrated heating and massage of a body portion of the user as central circumferential portion 186 applies relatively larger pressure on the body of the user. Although, the first stone 102 having two heaters 180, 184 is shown and contemplated, it may be appreciated that the first stone 102 may include a single heater, for example, the first heater 180 or the second heater 184.

Also, the first heater 180 and the second heater 184 are communicatively coupled to the controller 140 via the first and second communication devices 142, 144, and the controller 140 controls the operation of the first heater 180 and the second heater 184 to maintain the temperature of the first stone 102 within a desired temperature range or at a selected temperature selected by a user using the first selector 126. Also, the second communication device 144 is mounted to the frame 150 and may be arranged inside the receptacle 166. As the battery 170 is arranged inside the first stone 102, a need for external power source to heat the stone is eliminated, enhancing the portability and case of use, catering to massage therapists and individuals alike.

Further, referring back to FIG. 4, the first stone 102 includes a plurality of indicator lights 200 arranged arrayed circumferentially around the first wall of the frame. As shown, to retain the indicator lights, an inner surface 202 of the first wall 160 defines a plurality of cavities 204 arrayed circumferentially around the first wall 160, and the indicator lights 200 are arranged inside the cavities 204. In some embodiments, the first wall 160 includes either at least one hole or a transparent portion corresponding to each cavity 204 to enable the illumination of the first stone 102 in response to one or more operating parameters or operating conditions of the first stone 102.

For example, the controller 140 is configured to control and actuate the indicator lights 200 such that the indicator lights 200 emit light of a first color, for example, solid green color upon completion of a charging of the at least one battery 170. Further, the controller 140 controls the indicator lights 200 during charging of the at least one battery 170 to cause the blinking of the indicator lights 200. In the embodiment, the blinking indicator lights 200 emit green color. Additionally, the controller 140 is configured to blink the indicator lights 200 at a plurality of colors when a charging level of the at least one battery 170 is low i.e., below a threshold value to prompt the user to charge the at least one battery 170 of the first stone 102. It may be appreciated that the at least one battery 170 of the first stone 102 is charged when the first stone 102 is arranged inside the first cavity 116. In some embodiment, the system 100 may include a wireless charging unit, known in the art, arranged inside the casing 108 to charge the at least one battery 170 of the first stone 102 when the first stone 102 is arranged inside the first cavity 116. Alternatively, the first stone 102 may include electric connectors that contact the electrical connectors disposed inside the first cavity 116 to enable the charging of the at least one battery 102. Moreover, the system 100 may include a master battery arranged inside the casing 108 to enable charging of the batteries of the stones 102, 104. It may be envisioned that the master battery may be a rechargeable battery.

In some embodiment, the controller 140 is arranged to cause the illumination of the indicator lights 200 at a second color different from the first color when the massage system 100 is started/switched on. In some embodiments, the system 100 may include an ON-OFF switch to switch on/start and switch off or shutdown the system 100. Also, the controller 140 is configured to actuate the indicator lights 200 at a third color, for example, yellow, when the temperature of the first stone is at a low level i.e. in a first temperature range. Further, the controller 140 is configured to actuate the indicator lights 200 at a fourth color, for example, red when the temperature of the first stone 102 is at a high level i.e. in a second temperature range. Further, the controller 140 is configured to actuate the indicator lights 200 at a fifth color, for example, orange when the temperature of the first stone 102 is at an intermediate level i.e. in third second temperature range that lies between the maximum of the first temperature range and the minimum of the second temperature range. In this manner, the controller 140, by actuating the indicator lights 200 at different colors, indicates the user the temperature of the first stone 102

In some embodiments, the controller 140 may include a processor and a memory. The memory may be integrated into the controller 140, but those skilled in the art will understand that the memory may be separate from the controller but onboard the system 100, and/or remote from the system 100 and the controller 140, while still being associated with and accessible by the controller 140 to store information in and retrieve information from the memory as necessary during the operation of the system 100. For example, the identification codes corresponding to the stones 102, 104 are stored inside the memory. It may be appreciated the identification code associated with the each of the stones 102, 104 are received and stored inside the memory when the stones 102, 104 are paired with the cavities 116, 118 for the first time.

Although the processor is contemplated, it is also possible and contemplated to use other electronic components such as a microcontroller, an application specific integrated circuit (ASIC) chip, or any other integrated circuit device. The processor is configured to execute specified instructions, which controls and monitors various functions associated with the stones 102, 104, the sensors, 130, 132, the displays 120, 122, the selectors 126, 128, etc.

Like numbers refer to like elements throughout. Thus, the same or similar numbers may be described with reference to other drawings even if they are neither mentioned nor described in the corresponding drawing. Also, elements that are not denoted by reference numbers may be described with reference to other drawings.

Many modifications and other implementations of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific implementations disclosed and that modifications and other implementations are intended to be included within the scope of the appended claims. Moreover, although the foregoing descriptions and the associated drawings describe example implementations in the context of certain example combinations of elements and/or functions, it should be appreciated that different combinations of elements and/or functions may be provided by alternative implementations without departing from the scope of the appended claims.

What is claimed is:

1. A massage system, comprising:
a case defining at least one cavity;
at least one stone removably arranged inside the at least one cavity, wherein the at least one stone includes
a housing,
at least one heater arranged inside the housing and adapted to heat at least a portion of the housing, and
at least one battery disposed inside the housing and electrically coupled to the at least one heater to power the at least one heater; and
a controller mounted to the case and adapted to control the at least one heater to achieve a desired heating of the at least one stone.

2. The massage system of claim 1, wherein the at least one heater includes a first heater arranged inside the housing to heat a flat first surface of the housing.

3. The massage system of claim 2, wherein the first heater is a ceramic heater having a ceramic plate.

4. The massage system of claim 2, wherein the at least one heater includes a second heater arranged inside the housing to heat a central circumferential surface of the housing.

5. The massage system of claim 4, wherein the second heater is a coil heater including a coil heating element.

6. The massage system of claim 1 further comprising at least one display mounted to the case and communicatively coupled to the controller and configured to display a temperature of the at least one stone.

7. The massage system of claim 1 further comprising at least one selector associated with the at least one stone and configured to enable a user to select a desired temperature of the at least one stone, wherein the controller controls the at least one heater based on an input received from the at least one selector.

8. The massage system of claim 1 further comprising a first communication device mounted to the case and a second communication device arranged inside the housing of the at least one stone to enable a data transfer between the controller and the at least one stone to enable the controller to control of the at least one stone.

9. The massage system of claim 1, wherein the at least one stone includes a plurality of indicator lights mounted to the housing and configured to be actuated, by the controller, based one or more operating conditions of the at least one stone.

10. The massage system of claim 9, wherein the controller actuates and control the indicator lights to emit one or more colors based on the one or more operating conditions of the at least one stone.

11. The massage system of claim 1, wherein the housing includes a frame supporting the at least one battery and the at least one heater and a pair of covers coupled to the frame.

12. The massage system of claim 11, wherein each of the pair of covers is removably engaged to the frame.

13. The massage system of claim 11, wherein the frame includes
a base,
a first wall extending in a first direction from the base and defining a receptacle to receive the at least one battery, and
a second wall extending in a second direction, opposite to the first direction, from the base and defining a compartment to receive and support the at least one heater.

14. The massage system of claim 13, wherein a first cover of the pair of covers is arranged covering the receptacle, and a second cover of the pair of covers is arranged covering the compartment.

15. The massage system of claim 14, wherein
a first heater of the at least one heater is arranged partially inside the compartment and extends outwardly of the compartment to heat a flat first surface of the second cover, and
a second heater of the at least one heater is wrapped circumferentially around an outer surface of the second wall to heat a central circumferential surface of the housing.
